(19) United States
(12) Patent Application Publication
(10) Pub. No.: US 2025/0077857 A1
(43) Pub. Date: Mar. 6, 2025

(54) METHOD TO GENERATE SAFE NATURAL LANGUAGE MEDICAL REPORTS FOR DISEASE CLASSIFICATION

(71) Applicant: NEC Laboratories Europe GmbH, Heidelberg (DE)

(72) Inventors: Zhao XU, Heidelberg (DE); David FRIEDE, Heidelberg (DE)

(21) Appl. No.: 18/398,328

(22) Filed: Dec. 28, 2023

Related U.S. Application Data
(60) Provisional application No. 63/535,327, filed on Aug. 30, 2023.

(51) Int. Cl. G06N 3/08

(52) U.S. Cl. CPC G06N 3/08; G16H 50/70

(57) ABSTRACT

The present invention provides a computer-implemented, machine learning method for generating safe text. A first portion of a trainable prompt is generated using negative influential features and positive influential features of a predicted condition. A second portion of the trainable prompt is trained to steer a pre-trained large language model (PLLM) to generate the safe text using at least the first portion of the trainable prompt. The method has applications including, but not limited to, use cases in medicine (e.g., digital medicine, personalized healthcare, AI-assisted drug or vaccine development, diagnosis or treatment, disease prediction, etc.), and cyber security.

### CROSS-REFERENCE TO PRIOR APPLICATION

Priority is claimed to U.S. Provisional Application Ser. No. 63/535,327 filed on Aug. 30, 2023, the entire contents of which is hereby incorporated by reference herein.

### FIELD

The present invention relates to Artificial Intelligence (AI) and machine learning (ML), and in particular to medical AI and a method, system, data structure, computer program product, data structure, computer program product and computer-readable medium for generating safe text, having applications to medical reports for medical use cases such as disease classification.

### SUMMARY

In an embodiment, the present invention provides a computer-implemented, machine learning method for generating safe text. A first portion of a trainable prompt is generated using negative influential features and positive influential features of a predicted condition. A second portion of the trainable prompt is trained to steer a pre-trained large language model (PLLM) to generate the safe text using at least the first portion of the trainable prompt. The method has applications including, but not limited to, use cases in medicine (e.g., digital medicine, personalized healthcare, AI-assisted drug or vaccine development, diagnosis or treatment, disease prediction, etc.), and cyber security.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates a method and system for Large Language Model (LLM)-based medical report generation;

FIG. 2 schematically illustrates a method and system for safe LLM-based medical report generation according to an embodiment of the present invention;

FIG. 3 illustrates an example of attribute-based XAI with positive and negative features identified from tabular data as explanations to a disease prediction of a patient;

FIG. 4 illustrates a flow diagram of a method for safe LLM-based medical report generation according to an embodiment of the present invention; and

FIG. 5 is a block diagram of an exemplary processing system, which can be configured to perform any and all operations disclosed herein.

### DETAILED DESCRIPTION

Embodiments of the present invention provide an approach to adapt a pre-trained large language model to steer it to generate safe medical reports conditioned on facts identified by explainable AI (XAI) from patient data. The approach trains a soft prompt consisting of a finite number of trainable continuous vectors to control the text generation in a safe scope. In addition to providing to be able to generate more accurate and safe reports, the approach is also data- and resource-efficient, thereby conserving computational resources.

Medical reports contain comprehensive summaries of a patient's clinical history including background information, examinations, treatment, doctor opinions and more. Traditionally, these reports have been written by doctors themselves, consuming their valuable time and resources. Large Language Models (LLMs) have the potential to support doctors in writing medical reports more efficiently. The input of the LLM (the prompt) for this could be created by a hard-coded prompt template with manually-selected patient data by doctors. The LLM would then output the final medical reports given the prompt.

Hard-coded prompt templates are templates that contain natural language (e.g., tasks and guides for the LLM) and placeholders for individual data inputs. The prompt templates could be written manually and used by all doctors for all patients. The placeholders could be filled with the patient's data, identified by doctors, enabling the LLM to write a medical report for a specific patient and use case. The patient data could be selected and hand-crafted by the doctor to contain all the important information needed by the LLM to generate the report.

While this approach would offer an efficient way to write medical reports, there are a number of technical problems to overcome in order to be able to safely rely on the output, including that there could be incorrect and unsafe text in the report and that the generated text could contain hallucinations of fact. Thus, it presents a problem especially regarding safety, which is important especially in the medical domain. The doctor lacks control over guiding and improving the automatic report generation process. For example, existing technology does not provide the capability for the doctor to provide feedback to the LLM if the generated report contains unsafe content. The LLM also does not have the technical capability to learn from past mistakes and will repeat them over and over. The doctor will need to recognize and fix the flawed generated outputs every time. Consequently, the doctor needs to spend significant time and resources to evaluate and correct every single generated report, often containing very similar or even the same errors as previous generated ones. Further, the generated medical reports cannot be individualized. Although each doctor has their own style, the LLM is limited by existing technology to always output in the same generic style.

In a first aspect, the present invention provides a computer-implemented, machine learning method for generating safe text. A first portion of a trainable prompt is generated using negative influential features and positive influential features of a predicted condition. A second portion of the trainable prompt is trained to steer a pre-trained large language model (PLLM) to generate the safe text using at least the first portion of the trainable prompt.

In a second aspect, the present invention provides the method according to the first aspect, wherein the second portion of the trainable prompt includes a number of embedding vectors that are unknown prior to training, and wherein the method further comprises: obtaining patient data; and predicting a disease as the predicted condition using explainable artificial intelligence (XAI) and the patient data, wherein the XAI provides the negative influential features and the positive influential features along with explanation values.

In a third aspect, the present invention provides the method according to the first aspect or second aspect, further comprising: receiving new negative influential features and new positive influential features; generating a new first portion of the trainable prompt using the new negative influential features and the new positive influential features; generating an input prompt using the new first portion of the trainable prompt and the second portion of the trainable prompt; and generating a medical report using the PLLM and the input prompt.

In a fourth aspect, the present invention provides the method according to any of the first to third aspects, further comprising: receiving corrected medical reports from a particular user; updating the second portion of the trainable prompt using the corrected medical reports to be personalized to the particular user; and generating new medical reports for the user using the updated second portion of the trainable prompt and the PLLM.

In a fifth aspect, the present invention provides the method according to any of the first to fourth aspects, wherein the embedding vectors are frozen for the PLLM.

In a sixth aspect, the present invention provides the method according to any of the first to fifth aspects, wherein training the second portion of the trainable prompt to steer the PLLM to generate the safe text further comprises: constructing the second portion of the trainable prompt with a set of embedding vectors that will be trained to steer the PLLM to generate safe reports; generating medical reports using the PLLM, the negative influential features, and the positive influential features for pseudo patients; receiving labels and corrections from users analyzing the medical reports to generate corrected medical reports; computing a loss for each training example including a pair of: a) a medical report from the PLLM; and b) a corrected medical report of the corrected medical reports; and optimizing the loss of the training data for the embedding vectors of the second portion of the trainable prompt to learn to steer the PLLM to generate the safe text.

In a seventh aspect, the present invention provides the method according to any of the first to sixth aspects, wherein optimizing the loss of the training data for the embedding vectors includes obtaining optimal values of the embedding vectors to lead the PLLM to fit the corrected medical reports of training examples.

In an eighth aspect, the present invention provides the method according to any of the first to seventh aspects, further comprising predicting a disease as the predicted condition using explainable artificial intelligence (XAI) and patient data by training an XAI model to predict the disease using the patient data, wherein the XAI model ranks the negative influential features and the positive influential features with influential scores.

In a ninth aspect, the present invention provides the method according to any of the first to eighth aspects, wherein generating the first portion of the trainable prompt using the negative influential features and the positive influential features includes mapping the negative influential features and the positive influential features to a text with templates and a verbalizer.

In a tenth aspect, the present invention provides the method according to any of the first to ninth aspects, wherein the verbalizer projects class labels of the negative influential features and the positive influential features to pairs of verbs, and wherein the templates are used to place the negative influential features and the positive influential features.

In an eleventh aspect, the present invention provides the method according to any of the first to tenth aspects, further comprising: generating an input prompt comprising the first portion of the trainable prompt and the second portion of the trainable prompt, wherein the first portion of the trainable prompt is provided to the PLLM as a cloze instruction of influential features that corresponds to the negative influential features and the positive influential features; and generating a report in response to providing the input prompt to the PLLM.

In a twelfth aspect, the present invention provides the method according to any of the first to eleventh aspects, wherein the report identifies a patient, a predicted disease, and an explanation of the negative influential features and the positive influential features from the predicted disease.

In a thirteenth aspect, the present invention provides the method according to any of the first to twelfth aspects, wherein the second portion of the trainable prompt includes a number of continuous vectors of a same size as embedding vectors of the PLLM, wherein the embedding vectors are initialized randomly prior to training.

In a fourteenth aspect, the present invention provides a computer system for generating safe text comprising one or more processors which, alone or in combination, are configured to perform a machine learning method for generating safe text according to any of the first to thirteenth aspects.

In a fifteenth aspect, the present invention provides a tangible, non-transitory computer-readable medium for generating safe text which, upon being executed by one or more hardware processors, provide for execution of a machine learning method according to any of the first to thirteenth aspects.

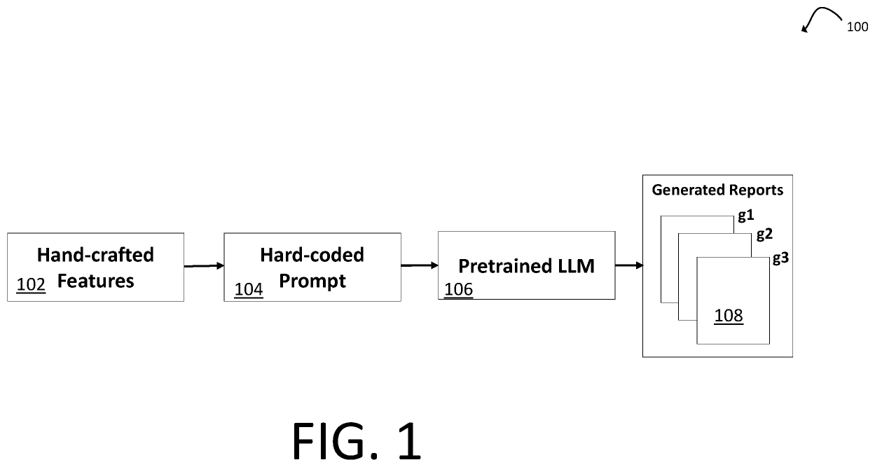

Referring to FIG. 1, the general operations of an LLM-powered medical report generator process (100) can be described as follows:
1. The patient's data is collected, selected, and pre-processed by the doctor to generate hand-crafted features 102.
2. The placeholders of the hard-coded prompt template 104 are filled with a hand-crafted selection of the patient's data 102.
3. The prompt 104 is sent to a pre-trained LLM 106 to generate the medical report 108.
4. The doctor reads, evaluates and corrects the generated medical report 108.

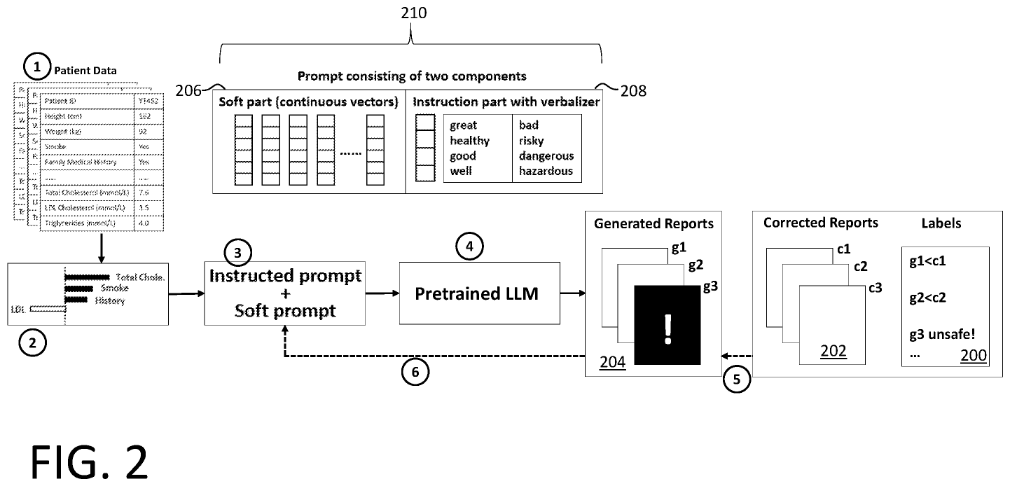

FIG. 2 shows a method and system according to an embodiment of the present invention that generates a safe, accurate, trustworthy, reliable and explainable medical report of a patient for a doctor to reduce resources, efforts and time, and to avoid critical mistakes resulting from incorrect data in LLM-generated text. The method and system provide to improve safety of the AI-generated medical reports, including the following enhancements to computer functionality:
Generating the text with a safety-aware training process to result in more accurate, safe and reliable reports.
Solving data privacy issues by publishing only soft prompts (continuous vectors).
Saving computational resources by data and resource efficiency: Only ˜2000 parameters to learn.

Each step of the method and system is detailed as follows:

### Step 2.1 (Represented as 1 in FIG. 2)—Obtaining Patient Data:

Historical patient data is obtained, for example, including:
General information, e.g., age, gender, weight, etc.
Symptoms, e.g., fever, pain, cough, nausea, etc.
Test results, e.g., systolic pressure, diastolic pressure, total cholesterol, LDL cholesterol etc.
Disease, e.g., Coronary Artery Atherosclerosis (CAD), type 2 diabetes, hypertension, etc.

The data can be obtained from a public dataset, or electronic health record (HER) databases.

Step 2.2 (Represented as 2 in FIG. 2)—Disease Prediction with Risky and Healthy Factors Using XAI:

The patient data is used to train an XAI model to predict a disease of a patient. The XAI will also provide explanations, such as the negative influential features and positive influential features, ranked with influential scores. For example, given a patient's data, an XAI model could predict the patient has a disease of Coronary Artery Atherosclerosis with a risk level of 76% and explain the prediction as:
Negative influential features: family medical history (value is Yes), smoking (value is Yes), and total cholesterol (value is 7.6 mmol/L).
Positive influential features: LDL cholesterol (value is 3.5 mmol/L).

XAI approaches such as Local Interpretable Model-Agnostic Explanations (LIME), KBLRN (see Alberto Garcia-Duran, Mathias Niepert, “KBLRN: End-to-End Learning of Knowledge Base Representations with Latent, Relational, and Numerical Features,” arXiv:1709:04676 (2018), which is hereby incorporated by reference herein) and gradient rollback (see U.S. Patent Application Publication No. 2022/0366274, which is hereby incorporated by reference herein) could be used to obtain the predictions and explanations.

### Step 2.3 (Represented as 3 in FIG. 2)—Trainable Mixture Prompt:

A trainable mixture prompt is constructed to be used as an input to a pre-trained large language model (PLLM). Embodiments of the present invention provide to learn the prompt to generate safe reports, which avoid hallucinations of fact. The prompt consists of two parts:
1. Soft part: A number K of continuous vectors, which dimension is the same as that of the embedding layer of the Pre-trained Large Language Model (PLLM). In embodiments, the number K of continuous vectors can be larger than or equal to 1 but can range between 10 and 100. A smaller number K continuous vectors corresponds to a faster training time whereas a larger number of K continuous vectors corresponds to more adaptability. The vectors are unknown, and will be trained during training process. This is known as a soft prompt (see Brian Lester, Rami Al-Rfou, Noah Constant, “The Power of Scale for Parameter-Efficient Prompt Tuning,” arXiv:2104:08691 (2021); and Xiang Lisa Li, Percy Liang, “Prefix-Tuning: Optimizing Continuous Prompts for Generation” arXiv:2101.00190 (2021), each of which is hereby incorporated by reference herein).
2. Instruction part: A mapping of the positive/negative influential features to a text with templates and a verbalizer. The verbalizer will project the class label of the influential features to pairs of meaningful verbs, such as (healthy, risky), (great, bad), (good, dangerous), etc. Templates are used to place the features. Some example templates can be: “can you generate a short medical note for the healthy features [xx][xx][xx] and the risky features [xx][xx][xx]?” and “Write a medical note to highlight the good features [xx][xx][xx] and the dangerous features [xx][xx][xx].” This is known as an instruction prompt (see Jason Wei, Maarten Bosma, Vincent Y. Zhao, Kelvin Guu, Adams Wei Yu, Brian Lester, Nan Du, Andrew M. Dai, Quoc V. Le, “Finetuned Language Models Are Zero-Shot Learners,” arXiv:2109.01652 (2022), which is hereby incorporated by reference herein).

Putting the two parts together, the input prompts to a PLLM according to an embodiment of the present invention can be, for example:
e₁ e₂ . . . e_{K} can you generate a short medical note for the healthy feature “LDL cholesterol: 3.5 mmol/L” and the risky features “family medical history:Yes”, “smoking:Yes”, “total cholesterol: 7.6 mmol/L” ?
e₁ e₂ . . . e_{K} write a short medical note to highlight the good feature “LDL cholesterol: 3.5 mmol/L” and the dangerous features “family medical history: Yes”, “smoking: Yes”, “total cholesterol:7.6 mmol/L”.

### Step 2.4 (Represented as 4 in FIG. 2)—Using the Pre-Trained Large Language Model (PLLM):

A Pre-trained Large Language Model (PLLM) is used to generate the text based on the input prompts. Advantageously, any other private or public PLLMs can be used. The model and its parameters will be frozen, and are not trained during the training process.

### Step 2.5 (Represented as 5 in FIG. 2)—Training the Prompt to Steer the PLLM for Generation of Safe Text:

Embodiments of the present invention provide a safety-aware training process to learn the trainable soft prompt. First, safe medical reports 200 are collected as training data. An LLM or PLLM can be used to generate some medical reports conditioned on the important features identified by XAI for different (pseudo) patients. The input prompts use the instruction part only. These reports, denoted as oᵢ, are labeled and corrected by medical professionals to be safe, in particular to match the truth of the domain knowledge and the facts of the patients. A corrected report 202 is denoted as cᵢ. Second, for each training example comprising a pair of a LLM-generated report 204 and a corrected report 202 by the medical professionals, embodiments of the present invention use the full prompt to generate a report, denoted as gᵢ, and compute loss as:

$${L}_{i} = {L}_{i , mle} + D ( {g}_{i} , {c}_{i} ) - D ( {g}_{i} , {o}_{i} )$$

where L_{i,mle} is any commonly used maximal likelihood loss of a generated text. The term D(gᵢ, cᵢ) specifies the distance between the generated text with the full prompt, and the corrected one. They should be as close as possible to provide that the generated reports 204 are safe. In contrast, D(gᵢ, oᵢ) denotes the distance of the generated report 204 to the unsafe one, which should be smaller. Third, the loss of the entire training data is optimized with respect to the embedding vectors of the soft prompt. Optimization methods, such as Adafactor, can be used to find the optimal values of the shared embedding vectors by all training examples. Adafactor may refer to a stochastic optimization method that reduces memory usage while retaining empirical benefits of adaptivity. In embodiments the PLLM is frozen, but the soft part of the prompt has K vectors. The methods of the current disclosure will learn to update the K vectors to fit the corrected reports from doctors. In embodiments the soft part of the trainable prompt is not frozen but is optimized during a training period.
Step 2.6—Generating, for a New Patient, a Medical Report with the Trained Prompt:

After the training procedure, the soft part of prompt 206, in particular the number K of embedding vectors, has been learned. The learned embedding vectors are frozen, and used by all prompts, which steers the PLLM to generate safe reports. For a new patient, given the positive and negative influential features 208, the prompt is constructed with the learned embedding vectors followed by the cloze instruction of influential features. With the prompt input 210, the PLLM will generate a safe report.

### Step 2.7—Training Personalized Prompts:

It is provided according to an embodiment of the present invention to personalize or individualize the prompts, for example to fit different individual styles of individual doctors. In particular, since every doctor has their own background, preference and style in writing medical reports for their patients, an embodiment of the present invention provides a personalization mechanism in a data- and resource-efficient manner. To do so, a global soft prompt is trained with generic corrected reports. Then, for each doctor, a single copy of the global soft prompt is obtained. Next, the corrected medical reports by a particular doctor are collected. After that, the soft prompt is fine-tuned with the doctor-specific reports. Finally, a personalized soft prompt will be learned, which will steer the PLLM to generate the medical report preferred by the doctor. In embodiments the soft prompt can be fine-tuned by changing the soft prompt slightly in the direction of the corrected reports (e.g., doctor-specific reports) and away from the generated ones—back-propagation via the loss function. Personalization of the soft prompt can be performed for one user or entity such as a doctor or a hospital. This provides to further reduce the effort and time, and required resources, of each single doctor. Also, since this personalization mechanism only trains and stores a soft prompt for each doctor, which consists of a limited number of embedding vectors, the training and storage is computational resource friendly.

Embodiments of the present invention thus provide for general improvements to computers in machine learning systems to provide an LLM system capable of generating text that is safer, more trustworthy, reliable and accurate, as well as explainable and personalizable. Although an especially advantageous use of the improved LLM system is for medical report generation, embodiments of the present invention also apply for the generation of other text for other machine learning tasks in other technical domains. Thus, in addition to the improvements in computer functionality in an AI LLM-based system to generate safe and correct text, embodiments of the present invention enable improvements also in the technical fields of application through improved, safer and more reliable decision-making and improved optimization of the machine learning task at hand that uses the generated text. Exemplary use cases in medical AI include disease prediction, digital medicine, personalized healthcare, and AI-assisted drug or vaccine development, etc., and generating cyber-attack reports. For example, collected data can be properties of activities, e.g., time stamp, source and destination IPs, source and destination ports, protocols. The AI-LLM-based system can generate reports that detail why a user's behavior can be an attack on a system. Negative influential factors can include the most suspicious factors such as a source IPs.

In an exemplary embodiment, the present invention can be applied for medical report generation for disease prediction for patients. Besides predictions, the explanations can also be provided to increase the trust of the doctors in the AI system. Since the doctors have no machine learning background, embodiments of the present invention advantageously provide that the explanations can be represented by natural language with some domain knowledge. In addition, since each doctor serves many patients per day, the automatic generation of medical reports in a manner still controlled by doctors and conditioned on the predictions, explanations and domain-specific knowledge, enables to significantly reduce the efforts, time and resources of doctors. To this end, embodiments of the present invention can generate safe medical reports for doctors based on AI-driven predictions and explanations, as well as domain knowledge retained in the LLM system. The data source for this use case includes patient data about symptoms and test results. Corrected medical reports by the doctors of the customer hospitals can also be used if the doctors request personalized reports fitting their background and preferences specifically. Application of the method according to an embodiment of the present invention generate safe medical reports based on AI-driven disease predictions and explanations. The output is safe, reliable, trustworthy, accurate and explainable, and possibly also personalized, medical reports on disease predictions for patients.

In another exemplary embodiment, the present invention can be applied to reduce unsafe generations in medical reports, for example for heart diseases. Heart diseases can be caused by multiple distinct factors. Usually, each patient presents a unique case requiring an individual analysis, evaluation, and medical report. The following steps 1-5 are performed.

### Step 1—Collecting Patient Data:

The patient's data consists of general information, symptoms, and the test results, for example:

| | |
|---|---|
| Gender | Male |
| Smoker | Yes |
| Family history | Father with heart disease |
| Symptoms | Chest Pain |
| Systolic pressure | normal |
| Total cholesterol | High |
| LDL cholesterol | High |

### Step 2—Predicting Diseases and Risk Factors Using XAI:

XAI methods such as Decision Trees can not only predict the patient's condition such as a specific heart disease, but they can also provide explanation values for each factor within the patient's data that might have been influential in making the prediction, for example, the factors and explanation values could be:

| | |
|---|---|
| Total Cholesterol | 89 |
| Smoker | 65 |
| Family history | 55 |
| Gender | 8 |
| Systolic pressure | −1 |
| LDL cholesterol | −42 |

### Step 3—Construct the Trainable Prompt:

The prompt template contains not only placeholders for the XAI factors, but also an instruction part, the verbalizer that projects pairs from positive to negative class labels to describe the influential factors. In embodiments, the explanation values above can be used to rank the XAI factors. The top K factors with the largest/smallest values are selected as the positive/negative influential factors. In embodiments, positive influence features can be defined as features which are good for patient health, and negative features can be defined as features which are risky for patient health. The words such as risky, dangerous, bad, negative, can serve as candidates for verbalizations of negative influential features, whereas words such as healthy, good, and positive are candidates for verbalizations of positive influential features. Different verbalizations can have a different effect in steering the language models. The model is trained to find the optimal verbalizations. For example:
“You are a helpful Chatbot and support with writing medical reports. You answer truthfully and if there is something you don't know, you say so. Do not make information up.
Your task is to generate a short medical note to highlight the [healthy] features such as [High LDL cholesterol] and the [risky] features such as [Total cholesterol, is smoking, Father with heart disease].”

Furthermore, the prompt also includes a number, e.g., 8 continuous vectors of the same size as the embedding vectors of the LLM, e.g., 256. The vectors are initialized randomly and will be trained during the training procedure. For example:
1. [0.8923, −0.1332, . . . , 1.2342]
2. . . .
8. [−0.353, 0.7309, . . . , −1,0038]
   Step 4—Input the Prompt into a PLLM to Generate a Medical Report:

A PLLM is used to generate the medical report based on the input prompt. The medical report might be flawed or contain unsafe content, for example:

Medical Report for patient XXX
. . .
Patient XXX suffers from heart disease ABC. The patient's history with smoking might be one of the factors for their condition.
. . .
The healthy influence of the high LDL value suggests that the patient should generally consume more fats.
. . .

### Step 5—Train the Prompts to Steer the PLLM to Generate Safe Medical Reports:

After collecting a training set containing generated medical reports with safe/unsafe flags together with corrected versions of the reports, the training set is used to train the soft prompt to stop generating unsafe content and to mimic the corrected medical reports, for example:
“The healthy influence of the high LDL value suggests that the patient should generally consume more fats.”—Unsafe
   Reduce probability of using healthy for positive influences of the factors.
   Change the 8 vectors of the prompt to reduce the probability of generating this output.
   Change the 8 vectors of the prompt to increase the probability of generating output similar to the correct versions.

In an embodiment, the present invention provides a method to generate safe medical reports for disease predictions or other machine learning tasks in medical AI, the method comprising the steps of:
1) Patient data collection (Step 2.1).
2) Disease prediction with risky and healthy factors using XAI (Step 2.2).
3) Construct a trainable prompt (Step 2.3).
4) Select a Pre-trained Large Language Model (PLLM) (Step 2.4).
5) Train the prompt to steer the PLLM for generation of safe texts (Step 2.5).
6) For a new patient, generate a medical report with the trained prompt (Step 2.6).
7) Where personalization is to be provided, train a personalized prompt for a particular doctor to fit their style (Step 2.7).

Embodiments of the present invention provide for the following improvements and technical advantages over existing technology:
1) Automatically producing a safe natural language medical report on disease classification enhancing computer functionality of the AI LLM-based system to be more safe, accurate, trustworthy and reliable, as well as explainable, and provides to save both computational and medical resources.
   a. Constructing a trainable mixture prompt consisting of unknown embedding vectors (to be learned), verbalized indicators for positive and negative influences with instructions, and patient-specific disease prediction and important attributes.
   b. Constructing a safety-oriented loss to lead the generated report to be safe, which penalizes the medical reports that are steered by the trainable mixture prompt based on the distances to the safe and unsafe reports: penalize if it is far from the correct reports or close to the reports labeled as unsafe.
2) Providing a data- and resource-efficient method by only optimizing the trainable mixture prompt towards safety (only ˜2000 parameters to learn).
3) Generating texts with a safety-aware training process.
4) Solving data privacy concerns by publishing only soft prompts, which are continuous vectors and maintain the privacy of patient input data.

Safety is a critical issue in LLMs (see Derek Tam, Anisha Mascarenhas, Shiyue Zhang, Sarah Kwan, Mohit Bansal, Colin Raffel, “Evaluating the Factual Consistency of Large Language Models Through News Summarization,” arXiv:2211.08412 (2023); and Junyi Li, Xiaoxue Cheng, Wayne Xin Zhao, Jian-Yun Nie, Ji-Rong Wen, “HaluEval: A Large-Scale Hallucination Evaluation Benchmark for Large Language Models,” arXiv:2305.11747 (2023), each of which is hereby incorporated by reference herein). For medical report generation, existing technology does not address the safety issue (see Omar Alfarghaly, Rana Khaled, Abeer Elkorany, Maha Helal, Aly Fahmy, “Automated radiology report generation using conditioned transformers,” Informatics in Medicine Unlocked, Vol. 24 (2021); and ZhihongChen, YanSong, Tsung-HuiChang, XiangWan, “Generating Radiology Reports via Memory-driven Transformer,” arXiv:2010.16056 (2020), each of which is hereby incorporated by reference herein). The generated medical report can be hallucinated (thereby containing factually incorrect information), which largely reduces the trust of end users (doctors). Although additional information, e.g., retrieved documents and facts in a knowledge base, can improve faithfulness of the generated texts, there still exists a risk of fact hallucinations and generation of incorrect and unsafe information. Embodiments of the present invention provide to overcome these technical problems by providing a data- and computation-efficient solution, which trains a mixture prompt (˜2000 parameters) with a safety-oriented loss to adapt and steer LLMs to generate truthful texts. An embodiment of the present invention can be especially advantageously applied to generate safe medical reports in a data- and computation-efficient manner via a trainable mixture prompt and safety-oriented training process.

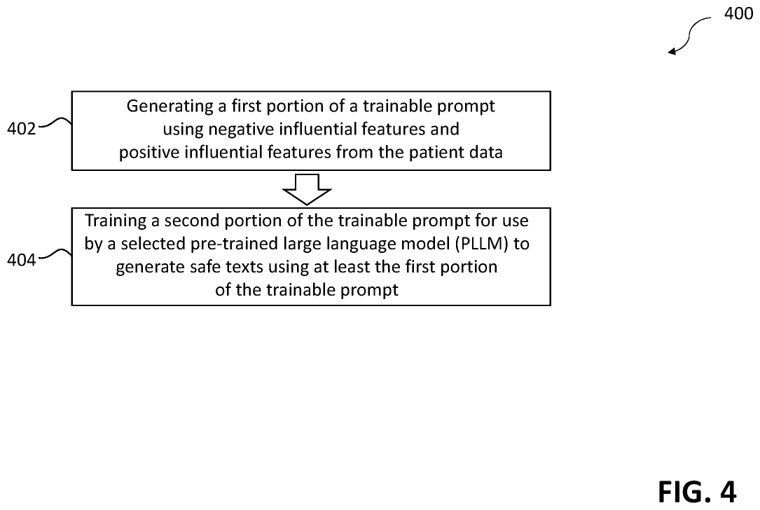

FIG. 4 illustrates a flow diagram 400 of a method for safe LLM-based medical report generation according to an embodiment of the present invention. The flow diagram 400 includes generating a first portion of a trainable prompt using negative influential features and positive influential features from patient data (see step 402). In embodiments, the first portion of the trainable prompt may be referred to as an instruction prompt. The flow diagram 400 includes training a second portion of the trainable prompt for use by a selected pre-trained large language model (PLLM) to generate safe texts using at least the first portion of the trainable prompt (see step 404). In embodiments, the second portion of the trainable prompt may be referred to as a soft prompt. The two prompts (instruction prompt and soft prompt) may form an input prompt that is provided to the PLLM to generate a safe report (e.g., medical report without hallucinations or other unsafe language). In embodiments, the patient data may include current and historical patient data for generation information of the patient, symptoms for the patient, test results for any tests run for the patient, or diseases associated with the patient or diagnosed for the patient. The patient data may be obtained from public data sources or from electronic health records (EHR) databases. In embodiments, the patient data may be used by explainable artificial intelligence (XAI) to predict disease. The XAI may generate the negative influential features and positive influential features while predicting the disease using the patient data.

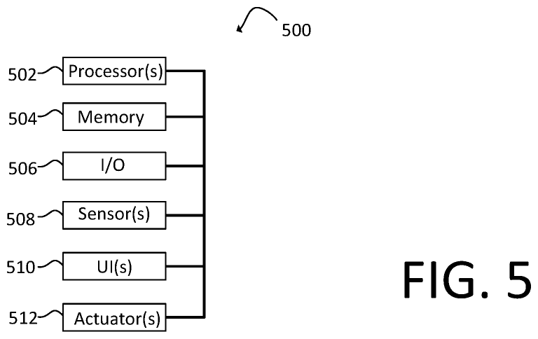

Referring to FIG. 5, a processing system 500 can include one or more processors 502, memory 504, one or more input/output devices 506, one or more sensors 508, one or more user interfaces 510, and one or more actuators 512. Processing system 500 can be representative of each computing system disclosed herein.

Processors 502 can include one or more distinct processors, each having one or more cores. Each of the distinct processors can have the same or different structure. Processors 502 can include one or more central processing units (CPUs), one or more graphics processing units (GPUs), circuitry (e.g., application specific integrated circuits (ASICs)), digital signal processors (DSPs), and the like. Processors 502 can be mounted to a common substrate or to multiple different substrates.

Processors 502 are configured to perform a certain function, method, or operation (e.g., are configured to provide for performance of a function, method, or operation) at least when one of the one or more of the distinct processors is capable of performing operations embodying the function, method, or operation. Processors 502 can perform operations embodying the function, method, or operation by, for example, executing code (e.g., interpreting scripts) stored on memory 504 and/or trafficking data through one or more ASICs. Processors 502, and thus processing system 500, can be configured to perform, automatically, any and all functions, methods, and operations disclosed herein. Therefore, processing system 500 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 500 can be configured to perform task “X”. Processing system 500 is configured to perform a function, method, or operation at least when processors 502 are configured to do the same.

Memory 504 can include volatile memory, non-volatile memory, and any other medium capable of storing data. Each of the volatile memory, non-volatile memory, and any other type of memory can include multiple different memory devices, located at multiple distinct locations and each having a different structure. Memory 504 can include remotely hosted (e.g., cloud) storage.

Examples of memory 504 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 504.

Input-output devices 506 can include any component for trafficking data such as ports, antennas (i.e., transceivers), printed conductive paths, and the like. Input-output devices 506 can enable wired communication via USB®, DisplayPort®, HDMI®, Ethernet, and the like. Input-output devices 506 can enable electronic, optical, magnetic, and holographic, communication with suitable memory 506. Input-output devices 506 can enable wireless communication via WiFi®, Bluetooth®, cellular (e.g., LTE®, CDMA®, GSM®, WiMax®, NFC®), GPS, and the like. Input-output devices 506 can include wired and/or wireless communication pathways.

Sensors 508 can capture physical measurements of environment and report the same to processors 502. User interface 510 can include displays, physical buttons, speakers, microphones, keyboards, and the like. Actuators 512 can enable processors 502 to control mechanical forces.

Processing system 500 can be distributed. For example, some components of processing system 500 can reside in a remote hosted network service (e.g., a cloud computing environment) while other components of processing system 500 can reside in a local computing system. Processing system 500 can have a modular design where certain modules include a plurality of the features/functions shown in FIG. 5. For example, I/O modules can include volatile memory and one or more processors. As another example, individual processor modules can include read-only-memory and/or local caches.

The following provides further background and description of exemplary embodiments of the present invention, which may overlap to some extent with some of the information provided above. To the extent the terminology used to describe the exemplary embodiments may differ from the terminology used to describe the above embodiments, a person having skill in the art would understand that certain terms correspond to one another in the different embodiments. Features described in the Attachment can be combined with features described above in various embodiments.

Explanations in machine learning are meaningful only if it is understandable for human users. In daily life, humans explain to each other in natural language. However, eXplainable AI (XAI) identifies, e. g., important attributes from tabular data as explanations to a prediction, which are often difficult for humans to understand, especially when they have no AI background. To solve this limitation, the current disclosure generates text-based comprehensive explanations conditioned on the important features selected by an XAI method. The explanations can borrow strength from XAI and Large Language Models (LLMs), integrating the concrete information extracted by XAI from tabular data and the general domain knowledge retained in LLM from large corpus.

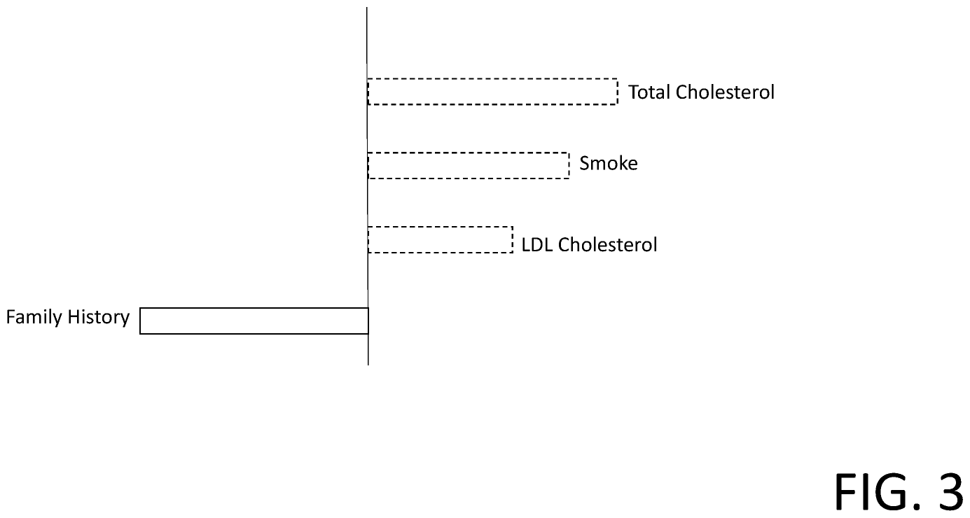

Explainable AI has become essential for AI-driven systems, as users request to know how a black-box AI system arrives at a prediction. For example, the off-the-shelf XAI tool LIME identifies important features from tabular data as explanations of a prediction. FIG. 3 depicts a typical example about disease prediction, where some of patient's attributes are selected as positive and negative features that cause the prediction. In FIG. 3, the positive patient attributes include family history (denoted by the solid black line bar), whereas the negative features that cause the prediction include total cholesterol, smoke, and LDL cholesterol (denoted by the dashed black line bars). However, such explanations are often difficult for users to understand, especially for users without AI background. In daily life, humans explain to each other in natural language, a question then naturally arises: can XAI improve understandability by explaining predictions in natural language given the identified important features?

Existing Natural Language Explanation (NLE) methods mainly focus on explaining natural language inference and image captioning. There is a lack of NLE methods to generate texts explaining predictions inferred by an XAI method from tabular data, which is a typical need in real world applications.

To solve this problem, the current disclosure provides users with text-based comprehensive explanations given the identified important features by XAI, such that the explanations can borrow strengths from both areas: integrate the concrete tabular data selected by XAI about how an AI black-box system arrives at a prediction; and the general domain knowledge of the prediction retained by LLMs from large corpus.

Two LLMs, MPT-7B and GPT3.5-175B, were studied to determine answers to questions related to problems implementing an XAI solution such as: can existing LLMs generate good text-based explanations given the identified features by XAI, if identified features by XAI conflict domain knowledge, can LLMs find this, are there hallucinations in the generated explanations by LLMs, which metrics can be used to measure the performance, which datasets can be used for benchmark, is human evaluation needed to test understandability of the text based explanations?Each LLM was used to generate natural language explanations conditioned on the identified features by XAI. In the studies GPT3.5-175B outperformed MPT-7B. The below text shows an example output of the two LLMs given the following crafted prompt: “Explain why the features cause the prediction. Predicted disease: Coronary Artery Atherosclerosis (risk 76%), Good features: no family history, Dangerous features: total cholesterol (7.6 mmol/L), Smoke (Yes), LDL cholesterol (3.5 mmol/L).”

Output by MPT-7B from the prompt above: I am not sure what you mean by “prediction”. If it is about risk assessment then I would say that this patient has an increased chance of developing coronary artery atherosclerosis in his lifetime but there are other factors which may influence whether or not he develops CAD during his life time e.g., smoking status etc.

Output by GPT3.5-175B from the prompt above: The prediction of a 76% risk of Coronary Artery Atherosclerosis (CAA) is based on a combination of good and dangerous features that are well-established risk factors for this condition. Let's break down why each of these features contributes to such a prediction:
1. No Family History (Good Feature): The absence of a family history of CAA is considered a positive feature because CAA can have a genetic component. If a patient has close relatives with CAA, it can suggest a genetic predisposition to the disease. The absence of family history indicates that genetic factors may not be as significant in this case, potentially making the condition more modifiable through lifestyle changes and risk factor management.
2. Total Cholesterol (7.6 mmol/L) and LDL Cholesterol (3.5 mmol/L) (Dangerous Features): Elevated levels of total cholesterol and LDL cholesterol are significant risk factors for CAA. Here's how they contribute to the predictions: . . .

With limited manual tests the GPT3.5-175B reported good results without showing obvious hallucinations. It even found conflict when the dangerous feature of smoke was modified from yes to no. Finding appropriate datasets as a benchmark was difficult as both tabular and text data are required. Recruiting human annotators to generate text-based explanations given the identified features by XAI can be expensive and time-consuming, especially when domain knowledge (such as medicine) is needed. A potential solution is to employ huge LLMs to generate labeled data. To reduce possible hallucinations, instruction-based self-validation can be used together with proper human-validation.

The current disclosure designs an initial framework based on prefix-tuning to train a NLE-specific LLM as described above in FIG. 2. In embodiments, an XAI method, such as LIME, is used to identify positive and negative important features from tabular data.

A task-specific LLM is trained according to prefix-tuning, which is data and computing efficient. In particular, an additional set of trainable embeddings are prepended to the instruction and the identified positive and negative important features, as shown in the below example:
Instruction: [v_{1,1} . . . v_{1,n}_{₁}] Given the predicted disease, good features and dangerous features, generate a short description.
Input:
[v_{2,1} . . . v_{2,n}_{₂}] Predicted disease: Coronary Artery Atherosclerosis (risk 76%)
[v_{3,1}] . . . [v_{3,n}_{₃}] Good features: no family history
[v_{4,1}] . . . [v_{4,n}_{₄}] dangerous features: total cholesterol (7.6 mmol/L), Smoke (Yes), LDL cholesterol (3.5 mmol/L)

During a training process, the unknown embeddings are trained to fit the labeled data, i.e., the faithful text-based explanation conditioned on the important features. The current disclosure provides technical solutions to the technical problems presented by integrating XAI methods with medical data by improving the understandability of XAI using the strength of LLMs. This can generate text based on the identified features by XAI and provide proper domain knowledge to further help users understand the predictions.

### REFERENCES

Pepa Atanasova, Oana-Maria Camburu, Christina Li-oma, Thomas Lukasiewicz, Jakob Grue Simonsen, and Isabelle Augenstein, (2023), Faithfulness tests for natural language explanations, Proceedings of the 61st Annual Meeting of the Association for Computational Linguistics (Volume 2: Short Papers), pages 283-294, Toronto, Canada, Association for Computational Linguistics.
Oana-Maria Camburu, Tim Rocktaschel, Thomas Lukasiewicz, and Phil Blunsom, (2018), e-snli: Natural language inference with natural language explanations, Advances in Neural Information Processing Systems, volume 31, Curran Associates, Inc.
Miruna-Adriana Clinciu and Helen Hastie, (2019), A survey of explainable AI terminology, Proceedings of the 1st Workshop on Interactive Natural Language Technology for Explainable Artificial Intelligence (NL4XAI 2019), pages 8-13, Association for Computational Linguistics.
Marina Danilevsky, Kun Qian, Ranit Aharonov, Yan-nis Katsis, Ban Kawas, and Prithviraj Sen, (2020), A survey of the state of explainable AI for natural language processing, Proceedings of the 1st Conference of the Asia-Pacific Chapter of the Association for Computational Linguistics and the 10th International Joint Conference on Natural Language Processing, pages 447-459, Suzhou, China, Association for Computational Linguistics.
Geoffrey Hinton, Oriol Vinyals, and Jeff Dean, (2015), Distilling the knowledge in a neural network, arXiv preprint, arXiv:1503.02531.
Sawan Kumar and Partha Talukdar, (2020), NILE: Natural language inference with faithful natural language explanations, Proceedings of the 58th Annual Meeting of the Association for Computational Linguistics, pages 8730-8742, Online, Association for Computational Linguistics.
Brian Lester, Rami Al-Rfou, and Noah Constant, (2021), The power of scale for parameter efficient prompt tuning, Proceedings of the 2021 Conference on Empirical Methods in Natural Language Processing, pages 3045-3059, Online and Punta Cana, Domini-can Republic, Association for Computational Linguistics.
Xiang Lisa Li and Percy Liang, (2021), Prefix-tuning: Optimizing continuous prompts for generation, Proceedings of the 59th Annual Meeting of the Association for Computational Linguistics and the 11th International Joint Conference on Natural Language Processing (Volume 1: Long Papers), pages 4582-4597, Online, Association for Computational Linguistics.
Niels Mundler, Jingxuan He, Slobodan Jenko, and Mar-tin Vechev, (2023), Self-contradictory hallucinations of large language models: Evaluation, detection and mitigation, arXiv preprint, arXiv:2305.15852.
Marco Tulio Ribeiro, Sameer Singh, and Carlos Guestrin, (2016), “why should I trust you?” Explaining the predictions of any classifier, Proceedings of the 22nd ACM SIGKDD International Conference on Knowledge Discovery and Data Mining, San Fran-cisco, CA, USA, Aug. 13-17, (2016), pages 1135-1144.
Abdus Salam, Rolf Schwitter, and Mehmet Orgun, (2021), Generating and modifying natural language explanations, Proceedings of the 19th Annual Workshop of the Australasian Language Technology Association, pages 149-157, Online, Australasian Language Technology Association.
F. Sammani, T. Mukherjee, and N. Deligiannis, (2022), Nlx-gpt: A model for natural language explanations in vision and vision-language tasks, 2022 IEEE/CVF Conference on Computer Vision and Pattern Recognition (CVPR), pages 8312-8322, Los Alamitos, CA, USA, IEEE Computer Society.
Rohan Taori, Ishaan Gulrajani, Tianyi Zhang, Yann Dubois, Xuechen Li, Carlos Guestrin, Percy Liang, and Tatsunori B. Hashimoto, (2023), Stanford alpaca: An instruction-following llama model, <https://github.com/tatsu-lab/stanford_alpaca>.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A computer-implemented method for generating safe text, the computer-implemented method comprising:
generating a first portion of a trainable prompt using negative influential features and positive influential features of a predicted condition; and
training a second portion of the trainable prompt to steer a pre-trained large language model (PLLM) to generate the safe text using at least the first portion of the trainable prompt.

2. The computer-implemented method according to claim 1, wherein the second portion of the trainable prompt includes a number of embedding vectors that are unknown prior to training, and wherein the method further comprises:
obtaining patient data; and
predicting a disease as the predicted condition using explainable artificial intelligence (XAI) and the patient data, wherein the XAI provides the negative influential features and the positive influential features along with explanation values.

3. The computer-implemented method according to claim 2, further comprising:
receiving new negative influential features and new positive influential features;
generating a new first portion of the trainable prompt using the new negative influential features and the new positive influential features;
generating an input prompt using the new first portion of the trainable prompt and the second portion of the trainable prompt; and
generating a medical report using the PLLM and the input prompt.

4. The computer-implemented method according to claim 2, further comprising:
receiving corrected medical reports from a particular user;
updating the second portion of the trainable prompt using the corrected medical reports to be personalized to the particular user; and
generating new medical reports for the user using the updated second portion of the trainable prompt and the PLLM.

5. The computer-implemented method according to claim 2, wherein the embedding vectors are frozen for the PLLM.

6. The computer-implemented method according to claim 1, wherein training the second portion of the trainable prompt to steer the PLLM to generate the safe text further comprises:
constructing the second portion of the trainable prompt with a set of embedding vectors that will be trained to steer the PLLM to generate safe reports;
generating medical reports using the PLLM, the negative influential features, and the positive influential features for pseudo patients;
receiving labels and corrections from users analyzing the medical reports to generate corrected medical reports;
computing a loss for each training example including a pair of: a) a medical report from the PLLM; and b) a corrected medical report of the corrected medical reports; and
optimizing the loss of the training data for the embedding vectors of the second portion of the trainable prompt to learn to steer the PLLM to generate the safe text.

7. The computer-implemented method according to claim 6, wherein optimizing the loss of the training data for the embedding vectors includes obtaining optimal values of the embedding vectors to lead the PLLM to fit the corrected medical reports of training examples.

8. The computer-implemented method according to claim 1, further comprising predicting a disease as the predicted condition using explainable artificial intelligence (XAI) and patient data by training an XAI model to predict the disease using the patient data, wherein the XAI model ranks the negative influential features and the positive influential features with influential scores.

9. The computer-implemented method according to claim 1, wherein generating the first portion of the trainable prompt using the negative influential features and the positive influential features includes mapping the negative influential features and the positive influential features to a text with templates and a verbalizer.

10. The computer-implemented method according to claim 9, wherein the verbalizer projects class labels of the negative influential features and the positive influential features to pairs of verbs, and wherein the templates are used to place the negative influential features and the positive influential features.

11. The computer-implemented method according to claim 1, further comprising:
generating an input prompt comprising the first portion of the trainable prompt and the second portion of the trainable prompt, wherein the first portion of the trainable prompt is provided to the PLLM as a cloze instruction of influential features that corresponds to the negative influential features and the positive influential features; and
generating a report in response to providing the input prompt to the PLLM.

12. The computer-implemented method according to claim 11, wherein the report identifies a patient, a predicted disease, and an explanation of the negative influential features and the positive influential features from the predicted disease.

13. The computer-implemented method according to claim 1, wherein the second portion of the trainable prompt includes a number of continuous vectors of a same size as embedding vectors of the PLLM, wherein the embedding vectors are initialized randomly prior to training.

14. A computer system for generating safe text, the computer system comprising one or more hardware processors which, alone or in combination, are configured to provide for execution of the following steps:
generating a first portion of a trainable prompt using negative influential features and positive influential features of a predicted condition; and
training a second portion of the trainable prompt for use by a pre-trained large language model (PLLM) to generate the safe text using at least the first portion of the trainable prompt.

15. A tangible, non-transitory computer-readable medium having instructions thereon which, upon being executed by one or more processors, provide for generating safe text by execution of the following steps:
generating a first portion of a trainable prompt using negative influential features and positive influential features of a predicted condition; and
training a second portion of the trainable prompt for use by a pre-trained large language model (PLLM) to generate the safe text using at least the first portion of the trainable prompt.
